(19) United States
(12) Patent Application Publication
(10) Pub. No.: US 2022/0359075 A1
(43) Pub. Date: Nov. 10, 2022

(54) SYNTHESIS FOR RISK PREDICTION MODELS

(71) Applicant: International Business Machines Corporation, Armonk, NY (US)

(72) Inventors: Ze Fang Tang, Beijing (CN); Yuan Zhang, Beijing (CN); Yi Qin Yu, Beijing (CN); Jing Mei, Beijing (CN)

(21) Appl. No.: 17/315,853

(22) Filed: May 10, 2021

(51) Int. Cl. G16H 50/20; G16H 50/50; G16H 70/60

(52) U.S. Cl. CPC G16H 50/20; G16H 50/50; G16H 70/60

(57) ABSTRACT

This disclosure relates to a method, a system and a computer program product for synthesizing risk prediction models to generate a generalized risk prediction model for a particular disease. The method comprises retrieving a plurality of literatures from one or more databases. Each of the plurality of literatures defines a risk prediction model for a same disease. The method further comprises extracting study features from each of the plurality of literatures. The method further comprises extracting weights of risk factors in the risk prediction model defined by each of the plurality of literatures from the plurality of literatures. The method further comprises calculating adjusted weights of risk factors based on the extracted study features and the extracted weights of risk factors, to form an adjusted risk prediction model.

### BACKGROUND

The present invention relates to synthesis for risk prediction models, and more specifically, to a method, a system and a computer program product for synthesizing risk prediction models to generate a generalized risk prediction model for a particular disease.

Nowadays, risk prediction models for diseases can deliver knowledge and indicate risk factors of diseases for supporting the prevention and prognosis of diseases in the clinical settings. However, when searching the risk prediction models for a particular disease, people will generally get thousands of results. It will be a heavy labor work to synthesize these risk prediction models in medical papers or literatures together.

### SUMMARY

According to one embodiment of the present invention, there is provided a computer-implemented method. The computer-implemented method comprises retrieving a plurality of literatures from one or more databases. Each of the plurality of literatures defines a risk prediction model for a same disease. The computer-implemented method further comprises extracting study features from each of the plurality of literatures. The computer-implemented method further comprises extracting weights of risk factors in the risk prediction model defined by each of the plurality of literatures from the plurality of literatures. The computer-implemented method further comprises calculating adjusted weights of risk factors based on the extracted study features and the extracted weights of risk factors, to form an adjusted risk prediction model.

According to another embodiment of the present invention, there is provided a system. The system comprises one or more processors and a memory coupled to at least one of the one or more processors. The system further comprises a set of computer program instructions stored in the memory and executed by at least one of the one or more processors in order to perform action of retrieving a plurality of literatures from one or more databases. Each of the plurality of literatures defines a risk prediction model for a same disease. The system further comprises a set of computer program instructions stored in the memory and executed by at least one of the one or more processors in order to perform action of extracting study features from each of the plurality of literatures. The system further comprises a set of computer program instructions stored in the memory and executed by at least one of the one or more processors in order to perform action of extracting weights of risk factors in the risk prediction model defined by each of the plurality of literatures from the plurality of literatures. The system further comprises a set of computer program instructions stored in the memory and executed by at least one of the one or more processors in order to perform action of calculating adjusted weights of risk factors based on the extracted study features and the extracted weights of risk factors, to form an adjusted risk prediction model.

According to another embodiment of the present invention, there is provided a computer program product comprising a computer readable storage medium having program instructions embodied therewith. The program instructions are executable by a device to cause the device to perform a method. The method comprises retrieving a plurality of literatures from one or more databases. Each of the plurality of literatures defines a risk prediction model for a same disease. The method further comprises extracting study features from each of the plurality of literatures. The method further comprises extracting weights of risk factors in the risk prediction model defined by each of the plurality of literatures from the plurality of literatures. The method further comprises calculating adjusted weights of risk factors based on the extracted study features and the extracted weights of risk factors, to form an adjusted risk prediction model.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 depicts a cloud computing node according to an embodiment of the present invention.

FIG. 2 depicts a cloud computing environment according to an embodiment of the present invention.

FIG. 3 depicts abstraction model layers according to an embodiment of the present invention.

FIG. 4 shows example prediction performance of risk prediction models from three example papers.

FIG. 5 is a flowchart illustrating an exemplary method for synthesizing risk prediction models according to an embodiment of the present disclosure.

FIG. 6 shows a table that includes the extracted study features and the extracted weights of risk factors from n literatures.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

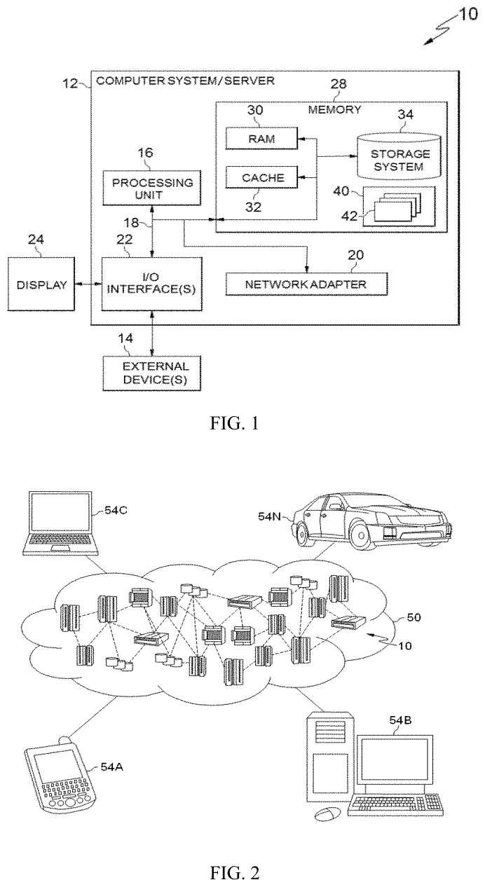

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12 or a portable electronic device such as a communication device, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

Referring now to FIG. 2, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

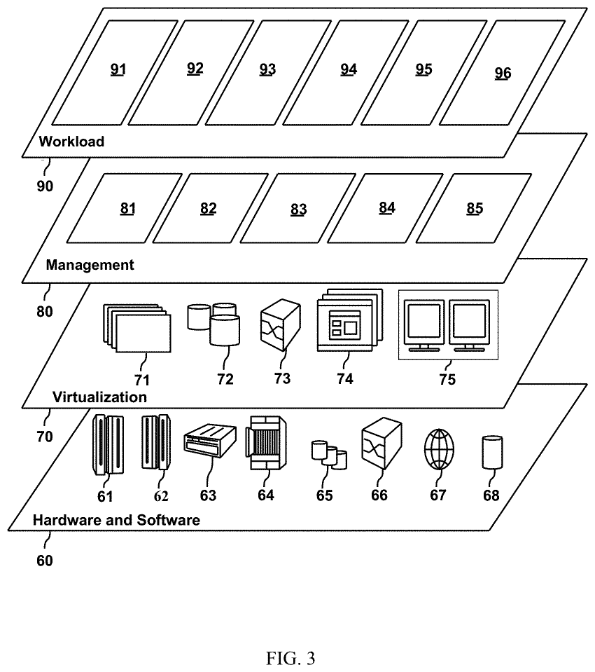

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and model synthesis 96.

The inventors of the present invention found that, even if risk prediction models are synthesized, the synthesized result is always qualitative, but not quantitative. Because of diverse populations, algorithms used in risk prediction models and diverse paper qualities, these risk prediction models usually demonstrate various risk factors, weights of risk factors, and prediction performance, which makes it harder for people to quantitatively synthesize these risk prediction models that are different in various aspects. In this disclosure, medical literatures may include medical papers or other medical documents including description of risk prediction models. For simplicity of explanation, literatures are also referred to as papers throughout this disclosure.

TABLE 1

| | Paper 1 | Paper 2 | Paper 3 |
|---|---|---|---|
| 1 | Age | Gender | Gender |
| 2 | Education level | Age | Age |
| 3 | Family history | Education level | Education level |
| | of diabetes | | |
| 4 | Smoking | Family history | Family history |
| | | of diabetes | of diabetes |
| 5 | Time for exercise | Smoking | Smoking |
| 6 | High blood pressure | Time for exercise | High blood pressure |
| 7 | Weight | High blood pressure | Weight |
| 8 | Height | Weight | Height |
| 9 | Waist circumference | Height | Waist circumference |
| 10 | | Waist circumference | Fasting blood glucose |
| 11 | | Fasting blood glucose | Triglycerides |
| 12 | | | High density |
| | | | lipoprotein cholesterol |
| 13 | | | Alanine |
| | | | aminotransferase |
| 14 | | | Glomerular filtration |
| | | | rate |

The above Table 1 shows three papers regarding risk prediction models for diabetes. As can be seen from Table 1, the three papers use different number of risk factors. For example, Paper 1 uses 9 risk factors in its risk prediction model; Paper 2 uses 11 risk factors in its risk prediction model; and Paper 3 uses 14 risk factors in its risk prediction model. In addition, different risk factors are considered by different papers. For example, the risk factor of “Triglycerides” is only considered by Paper 3, and Papers 1 and 2 do not consider this risk factor in their risk prediction models. Actually, there are totally fifteen different risk factors used in the three papers. Further, the algorithms used by the three papers may be different, and the prediction performance of risk prediction models of the three papers may be also different.

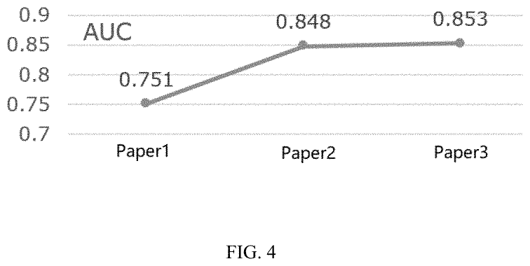

FIG. 4 shows example prediction performance of risk prediction models of the three example papers. The area under curve (AUC) of Papers 1 to 3 are 0.751, 0.848 and 0.853 respectively, as shown in FIG. 4. The AUC statistic is an empirical measure of classification performance based on the area under a receiver operating characteristic (ROC) curve. It evaluates the performance of a scoring classifier on a test. The AUC has a value between 0.5 and 1. The greater the AUC is, the more accurate the prediction of the model is. For example, in FIG. 4, Paper 3 has the best performance.

Based on the above differences among papers in various aspects, it is difficult to synthesize risk prediction models from different papers into a generalized risk prediction model that quantitatively takes into account various aspects of these papers. Thus, the inventors of the present invention propose a novel method for synthesizing risk prediction models to generate a generalized risk prediction model.

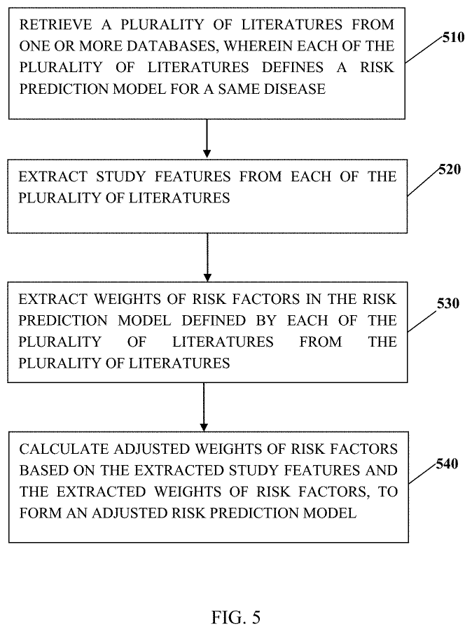

With reference now to FIG. 5, it is a flowchart illustrating an exemplary method for synthesizing risk prediction models according to an embodiment of the present disclosure.

It should be noted that the method for synthesizing risk prediction models according to embodiments of this disclosure could be implemented by computer system/server 12 of FIG. 1.

As shown in FIG. 5, at step 510, a plurality of literatures may be retrieved from one or more databases. According to one embodiment of the disclosure, the literatures are clinical or medical papers. Examples of the one or more databases are MEDLINE, PubMed and etc. Of course, other medical databases can be also searched as long as they are accessible. In one embodiment of the disclosure, a user may choose databases from a list of available databases to retrieve the plurality of literatures by selecting respective checkboxes of these databases. Each of the plurality of literatures may define a risk prediction model for a same disease. That is, the plurality of literatures is retrieved from databases with respect to a particular disease such as diabetes. Each of the plurality of literatures includes description of a risk prediction model for the particular disease.

For example, the plurality of literatures can be collected by using a Natural Language Processing (NLP) technology or a Named Entity Recognition (NER) technology. According to one embodiment of the disclosure, the literatures may be retrieved by using a searching method that assembles technologies such as PageRank and Topic-based clustering to accelerate the paper searching tasks.

The above specific searching method can be used in step 510 due to its advantages. However, other NLP algorithms or NER algorithms such as keyword searching could be also used to retrieve the plurality of literatures. No matter which technology is used, when the step 510 finishes, a lot of papers are collected. In one example, hundreds of papers are retrieved. Here, it assumes that n literatures or papers are retrieved and expressed as {p₁, . . . , pₙ}. Here, n is a number of retrieved literatures or papers and p is a variable used to indicate a literature or paper.

Referring back to FIG. 5, at step 520, study features are extracted from each of the plurality of literatures. According to one embodiment of the disclosure, the study features may include study features selected from the group consisting of: impact factor, prediction performance and sample size of a literature. The impact factor of a literature may be determined based on impact factor of the magazine or periodical that publishes the literature. The prediction performance of a literature may be determined based on the prediction performance (e.g., AUC) of the risk prediction model described in the literature. The sample size of a literature indicates the size of samples used in the literature. Normally, the bigger the sample size is, the better the literature is. Besides the above example study features, other study features of a literature may also be considered and extracted, such as authors' achievements or reputation.

For example, the study features can be extracted by analyzing the retrieved literature by using an NLP technology or an NER technology. According to one embodiment of the disclosure, the study features can be extracted by using an identification method. In this embodiment, a disease Named Entity Recognition (DNER) model and a PICO (Population, Intervention, Comparison, and Outcome) Classification Model may be established to extract and classify content of a paper. Here, the PICO framework is usually used to formulate evidence in the medical domain.

The above specific identification method can be used in step 520 due to its advantages. However, other NLP algorithms or NER algorithms such as keyword searching could be also used to extract the study features. No matter which technology is used, when the step 520 finishes, the study features defined by each of the plurality of literatures are extracted. Here, it assumes that d study features for each literature are extracted and expressed as {f₁, . . . , f_{d}}. Here, d is a number of extracted study features and f is a variable used to indicate a study feature.

According to one embodiment of the disclosure, d is the number of a full set of study features extracted from the plurality of literatures. However, sometimes, d study features cannot be extracted from every literature. For example, for Paper 1, the sample size might not be extracted since it does not recite this data. In this case, the element (e.g., f₃) in {f₁, . . . f_{d}} of Paper 1 that corresponds to sample size can be set to zero or a void value.

Referring back to FIG. 5, at step 530, weights of risk factors in the risk prediction model defined by each of the plurality of literatures are extracted from the plurality of literatures. For example, the above Table 1 has shown some examples of risk factors for diabetes.

For example, the weights of risk factors can be extracted by analyzing the retrieved literature by using an NLP technology or an NER technology. According to one embodiment of the disclosure, the weights of risk factors can also be extracted by using an identification method. In this embodiment, a DNER model and a PICO Classification Model may be established to extract and classify content of a paper.

The above specific identification method can be used in step 530 due to its advantages. However, other NLP algorithms or NER algorithms such as keyword searching could also be used to extract the weights of risk factors. No matter which technology is used, when the step 530 finishes, the weights of risk factors of each of the plurality of literatures are extracted. Here, it assumes that m weights of risk factors for each literature are extracted and expressed as {r₁, . . . , rₘ}. Here, m is a number of extracted weights of risk factors and r is a variable used to indicate a weight of a risk factor.

According to one embodiment of the disclosure, m is the number of a full set of study features extracted from the plurality of literatures. However, sometimes, m weights of risk factors cannot be extracted from every literature. Taking data in Table 1 as an example, because there are totally fifteen different risk factors used in the three papers, then m=15. However, for example, Paper 1 does not include the risk factor “Fasting blood glucose”, so this risk factor cannot be extracted from Paper 1. In this case, the element (e.g., r₁₁) in {r₁, . . . , rₘ} of Paper 1 that corresponds to “Fasting blood glucose” can be set to zero or a void value.

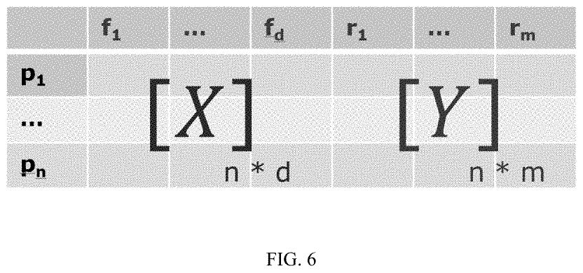

FIG. 6 shows a table that includes the extracted study features and the extracted weights of risk factors for the plurality of literatures. The table in FIG. 6 has n rows. The first row shows the study features {f₁, . . . f_{d}} and the weights of risk factors {r₁, . . . , rₘ} extracted from Paper p₁; and the last row shows the study features {f₁, . . . f_{d}} and the weights of risk factors {r₁, . . . , rₘ} extracted from Paper pₙ. From the table in FIG. 6, we can see that, all of the extracted study features form a n*d matrix X and all of the extracted weights of risk factors form a n*m matrix Y. As described above, n is a number of retrieved papers, d is a number of extracted study features, and m is a number of extracted weights of risk factors. That is, the matrix X includes study features extracted from the plurality of literatures and the matrix Y includes weights of all risk factors included in the plurality of literatures. The table in FIG. 6 include all information extracted from collected literatures. Then, the generalization will be performed as follows.

Referring back to FIG. 5, at step 540, adjusted weights of risk factors are calculated based on the extracted study features and the extracted weights of risk factors, to form an adjusted risk prediction model. The adjusted risk prediction model includes risk factors with the adjusted weights. That is, according to the present invention, a generalized risk prediction model is generated, which includes risk factors with weights adjusted quantitatively according to information extracted from a plurality of papers.

Specifically, according to one embodiment of the disclosure, calculating the adjusted weights of risk factors may comprise training a multi-task model using the extracted study features and the extracted weights of risk factors to obtain a coefficient matrix W that follows a Matrix Variate Normal (MVN) distribution. The multi-task model and the MVN distribution are described in detail in “LINKAGE: An Approach for Comprehensive Risk Prediction for Care Management” (Proceedings of the 21th ACM SIGKDD International Conference on Knowledge Discovery and Data Mining, August 2015, Pages 1145-1154). Specifically, the multi-task model that follows the MVN distribution can be expressed as model (1) as follows.

W_{d*m}˜MVN(0,Γ,Ω_{m*m}) (1)

Here, W_{d*m} is a d*m coefficient matrix of the multi-task model that is used to characterize risk association, i.e., association among risk factors. That is, the multi-task model is trained with data in the matrix X and the matrix Y to find a relationship among elements in the matrix Y. The main goal of the training is to reveal the relationships between study features and risk factors (learnt by the multi-task model), and finally get suggestions of how m risk factors contribute to the clinical problem based on the multi-task model. Additionally, it assumes that the risk association is revealed in the structure of the coefficient matrix W_{d*m}. In model (1), 0 is a d*m matrix of zeros. F is a d*d matrix, which represents the study feature-wise covariances of W. Ω is a m*m symmetric positive definite matrix, which represents the risk factor-wise covariances of W. According to one embodiment, the matrix Ω is used to indicate a relationship among m risk factors, and also referred to as association matrix.

According to one embodiment of the disclosure, calculating the adjusted weights of risk factors may further comprise decomposing the coefficient matrix W to obtain a matrix Ω representing a relationship between risk factors according to the MVN distribution. In order to obtain the matrix Ω, the coefficient matrix W is decomposed during the training according to the MVN distribution. When the training process of the multi-task model converges, it is supposed to obtain an optimized coefficient matrix W and accordingly a matrix Ω. That is, due to the MVN distribution, if the coefficient matrix Win model (1) is obtained, the matrix Ω can be obtained accordingly. According to one embodiment of the disclosure, calculating the adjusted weights of risk factors may further comprise calculating the adjusted weights of risk factors using the matrix Ω.

Here, a simple example is provided to explain the process of calculating the adjusted weights of risk factors. It assumes that the following coefficient matrix W_{d*m} and matrix Ω are obtained when the training process of the multi-task model converges. In this example, d=3 and m=5.

$${W}_{d * m} = [ \begin{matrix}0.12 & 0.45 & 0.23 & 0.42 & 0.89 \\ 0.18 & 0.32 & 0.76 & 0.46 & 0.02 \\ 0.42 & 0.24 & 0.56 & 0.62 & 0.08\end{matrix} ]$$

$${Ω}_{m * m} = [ \begin{matrix}1 & 0.45 & 0.23 & 0.42 & 0.89 \\ 0.45 & 1 & 0.24 & 0.45 & 0.02 \\ 0.23 & 0.24 & 1 & 0.62 & 0.08 \\ 0.42 & 0.46 & 0.62 & 1 & 0.04 \\ 0.89 & 0.02 & 0.08 & 0.04 & 1\end{matrix} ]$$

According to one embodiment of the disclosure, calculating the adjusted weights of risk factors using the matrix Ω may comprise accumulating elements of each row (or column) of the matrix Ω to obtain a vector and then dividing each element of the vector by a totally accumulated value of the matrix Ω to obtain a vector ω. Taking the above matrix Ω as an example, the following vector will be obtained by accumulating elements of each row (or column) of the matrix Ω: {2.99, 2.17, 2.17, 2.54, 2.03}. Additionally, when elements of the vector {2.99, 2.17, 2.17, 2.54, 2.03} are accumulated, a totally accumulated value of the matrix Ω will be 11.9. Then, if each element of the vector {2.99, 2.17, 2.17, 2.54, 2.03} is divided by the totally accumulated value of the matrix Ω (i.e., 11.9), that is, if normalization is performed, the vector ωₘ {0.25, 0.18, 0.18, 0.21, 0.17} is obtained.

Here, m elements of the vector ω correspond to the adjusted weights of risk factors. Then, the ω vector is used to build the logistic regression (LR) formula (2) as shown below.

$$L R : \frac{1}{1 + {e}^{- {\sum }_{1}^{m} {ω}_{i} {r}_{i}}} \qquad ( 2 )$$

According to one embodiment of the disclosure, the method for synthesizing risk prediction models may further comprise predicting a risk of the disease for a patient by using the adjusted risk prediction model. In the above formula (2), ωᵢ is an element (i.e., weight) from the vector ω and rᵢ is the risk factor corresponding to ωᵢ. That is, the logistic regression formula (2) including risk factors with the adjusted weights may be used to perform risk prediction of the particular disease for a patient.

Now, we turn to explain how to make the training of the multi-task model converge. According to one embodiment of the disclosure, training the multi-task model may comprise setting a loss function that considers variance within literatures and variance between literatures. The process of training the multi-task model may converge by minimizing the loss function.

As shown in FIG. 6, the study features extracted in step 520 may be expressed by a matrix X with n*d elements, here n is a number of the literatures retrieved in step 510, and d is a number of the extracted study features. Meanwhile, as shown in FIG. 6, the weights of risk factors extracted in step 530 may be expressed by a matrix Y with n*m elements, here n is a number of the literatures retrieved in step 510, and m is a number of the extracted weights of risk factors.

According to one embodiment of the disclosure, the loss function may be set as

min_{W}l(X,Y;W)+η+ϵ (3)

Here, minty means to solve a matrix W to cause the function l(X, Y; W) to be minimum, η is a term indicating the variance between literatures, and c is a term indicating the variance within literatures. The function l(X, Y; W) can be designed in many manners. According to one embodiment of the disclosure, for example, the function l(X, Y; W) can be determined as

l(X,Y;W)=Σᵢ₌₁ⁿΣⱼ₌₁^{m}½(y_{i,j}−Σₖ₌₁^{d}x_{i,k}*w_{k,j})² (4)

In the above equation (4), x_{i,k} represents elements from the matrix X, y_{i,j} represents elements from the matrix Y, and w_{k,j} represents elements from the coefficient matrix W. The above equation (4) is only an example of component of the loss function. Other loss function may be also applicable. For example, the sparsity patterns of w's could be used to reflect relatedness.

As to the terms η and ϵ in the above loss function (3), they are calculated based on the extracted weights of risk factors. According to one embodiment of the disclosure, the term η in the loss function may be calculated using the DerSimonian and Laird method. And the term ϵ in the loss function may be obtained by calculating an average of variances of weights of risk factors over literatures.

Taking the three papers shown in Table 1 as an example. It assumes that, the following weights of risk factors are extracted from Papers 1 to 3 in the above step 530. The weight in a cell of Table 2 corresponds to the risk factor in the cell at the same position of Table 1. For example, the weight “0.3” of Paper 1 in the first row of Table 2 corresponds to the risk factor “age” of Paper 1 in Table 1; the weight “0.6” of Paper 2 in the fourth row of Table 2 corresponds to the risk factor “Family history of diabetes” of Paper 2 in Table 1; and the weight “0.21” of Paper 3 in the last row of Table 2 corresponds to the risk factor “Glomerular filtration rate” of Paper 3 in Table 1. That is, the corresponding weights of risk factors in Table 1 are extracted and displayed in Table 2 with one-to-one correspondence.

TABLE 2

| | Paper 1 | Paper 2 | Paper 3 |
|---|---|---|---|
| 1 | 0.3 | 0.45 | 0.23 |
| 2 | 0.6 | 0.12 | 0.12 |
| 3 | 0.78 | 0.3 | 0.41 |
| 4 | 0.5 | 0.6 | 0.45 |
| 5 | 0.45 | 0.78 | 0.57 |
| 6 | 0.12 | 0.5 | 0.3 |
| 7 | 0.57 | 0.23 | 0.6 |
| 8 | 0.69 | 0.16 | 0.34 |
| 9 | 0.2 | 0.57 | 0.5 |
| 10 | | 0.69 | 0.78 |
| 11 | | 0.2 | 0.16 |
| 12 | | | 0.29 |
| 13 | | | 0.69 |
| 14 | | | 0.21 |

According to one embodiment of the disclosure, the variance within literatures may be expressed by:

$$ϵ = \frac{{\sum }_{i = 1}^{k} {V}_{i}}{k} \qquad ( 5 )$$

In the above equation (5), k is the number of papers, and Vᵢ=σ²/n. Here, σ² represents variance of weights of risk factors of a paper, and n represents the number of valid risk factors in the paper. Taking data in Table 2 as an example,

$$ϵ = ( {V}_{1} + {V}_{2} + {V}_{3} ) / 3 \\ = ( 0.05 / 9 + 0.052 / 11 + 0.041 / 14 ) / 3 \\ = ( 0.006 + 0.005 + 0.002 ) / 3 \\ = 0.004$$

The present invention is not limited to the above equation (5). Other calculation may be used as long as it takes into account variance of weights of risk factors within each retrieved paper. Meanwhile, according to one embodiment of the disclosure, the variance between literatures may be expressed by:

$$η = max \{ 0 , \frac{Q - df}{C} \} \qquad ( 6 )$$

In the above equation (6), df=k−1 and k is the number of papers. Other variables in equation (6) may be defined as follows.

$$C = {\sum }_{i = 1}^{k} {w}_{i} - \frac{{\sum }_{i = 1}^{k} {w}_{i}^{2}}{{\sum }_{i = 1}^{k} {w}_{i}} \qquad ( 7 )$$

$$Q = {\sum }_{i = 1}^{k} {{w}_{i} ( {Y}_{i} - {\overline{Y}}_{w} )}^{2} \qquad ( 8 )$$

In the above equations (7) and (8), w₁=1/Vᵢ. In the equation (8), Yᵢ represents sum of weights of risk factors of the ith paper and Y̅_{w} may be defined as follows.

$${\overline{Y}}_{w} = \frac{{\sum }_{i = 1}^{k} {w}_{i} {Y}_{i}}{{\sum }_{i = 1}^{k} {w}_{i}} \qquad ( 9 )$$

As to the example data in Table 2, we can have the follow results:

TABLE 3

| | | |
|---|---|---|
| Y₁ = 4.21 | Y₂ = 4.6 | Y₃ = 5.65 |
| w₁ = 181.381 | w₂ = 220.355 | w₃ = 555.311 |

With the calculated results in Table 3, the above equation (9) can be calculated to obtain Y̅_{w}=5.135. And in turn the above equation (8) can be calculated to obtain Q=365.547. Additionally, with the calculated results in Table 3, the above equation (7) can be calculated to obtain C=549.725. Finally, based on the values of Q and C, the above equation (6) can be calculated to obtain η=0.661.

The present invention is not limited to the above equations (6) to (9), which are based on the DerSimonian and Laird method. Other calculation may be used as long as it takes into account variance of weights of risk factors between retrieved papers. In the above example, after the above calculations, the loss function will become:

min_{W}l(X,Y;W)+0.661+0.004

With the above solution in the disclosure, different risk factors from different literatures are quantitively analyzed and importance of weights of these risk factors from different literatures is revealed. According to the present invention, a generalized risk prediction model can be generated despite the diverse risk factors, weights and prediction performance of hundreds of retrieved papers. In addition, for a given disease, the generalized risk prediction model including risk factors with adjusted weights can be used as a baseline risk model, which provides a study base for researchers of risk prediction models for a particular disease.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
retrieving, by one or more processing units, a plurality of literatures from one or more databases, wherein each of the plurality of literatures defines a risk prediction model for a same disease;
extracting, by the one or more processing units, study features from each of the plurality of literatures;
extracting, by the one or more processing units, weights of risk factors in the risk prediction model defined by each of the plurality of literatures from the plurality of literatures; and
calculating, by the one or more processing units, adjusted weights of risk factors based on the extracted study features and the extracted weights of risk factors, to form an adjusted risk prediction model.

2. The computer-implemented method according to claim 1, further comprising:
predicting, by the one or more processing units, a risk of the disease for a patient by using the adjusted risk prediction model.

3. The computer-implemented method according to claim 1, wherein calculating the adjusted weights of risk factors comprises:
training, by the one or more processing units, a multi-task model using the extracted study features and the extracted weights of risk factors to obtain a coefficient matrix W that follows a Matrix Variate Normal (MVN) distribution;
decomposing, by the one or more processing units, the coefficient matrix W to obtain a matrix Ω representing a relationship between risk factors according to the MVN distribution; and
calculating, by the one or more processing units, the adjusted weights of risk factors using the matrix Ω.

4. The computer-implemented method according to claim 3, wherein training the multi-task model comprises:
setting, by the one or more processing units, a loss function that considers variance within literatures and variance between literatures, wherein the process of training the multi-task model converges by minimizing the loss function.

5. The computer-implemented method according to claim 4, wherein:
the extracted study features are expressed by a matrix X with n*d elements, n is a number of the plurality of literatures, and d is a number of the extracted study features;
the extracted weights of risk factors are expressed by a matrix Y with n*m elements, n is a number of the plurality of literatures, and m is a number of the extracted weights of risk factors;
the loss function is set as min_{W}l(X, Y; W)+η+ϵ, wherein
l(X,Y;W)=Σᵢ₌₁ⁿΣⱼ₌₁^{m}½(y_{i,j}−Σₖ₌₁^{d}x_{i,k}*w_{k,j})²,
η is a term indicating the variance between literatures, ϵ is a term indicating the variance within literatures, x_{i,k} represents elements from the matrix X, y_{i,j} represents elements from the matrix Y, and w_{k,j} represents elements from the coefficient matrix W.

6. The computer-implemented method according to claim 5, wherein the term η in the loss function is calculated by using the DerSimonian and Laird method.

7. The computer-implemented method according to claim 1, wherein the study features include study features selected from the group consisting of: impact factor, prediction performance and sample size of a literature.

8. A system comprising:
one or more processors;
a memory coupled to at least one of the one or more processors;
a set of computer program instructions stored in the memory and executed by at least one of the one or more processors in order to perform actions of:
retrieving a plurality of literatures from one or more databases, wherein each of the plurality of literatures defines a risk prediction model for a same disease;
extracting study features from each of the plurality of literatures;
extracting weights of risk factors in the risk prediction model defined by each of the plurality of literatures from the plurality of literatures; and
calculating adjusted weights of risk factors based on the extracted study features and the extracted weights of risk factors, to form an adjusted risk prediction model.

9. The system according to claim 8, further comprising a set of computer program instructions stored in the memory and executed by at least one of the one or more processors in order to perform action of predicting a risk of the disease for a patient by using the adjusted risk prediction model.

10. The system according to claim 8, wherein calculating the adjusted weights of risk factors comprises:
training a multi-task model using the extracted study features and the extracted weights of risk factors to obtain a coefficient matrix W that follows a Matrix Variate Normal (MVN) distribution;
decomposing the coefficient matrix W to obtain a matrix Ω representing a relationship between risk factors according to the MVN distribution; and
calculating the adjusted weights of risk factors using the matrix Ω.

11. The system according to claim 10, wherein training the multi-task model comprises:
setting a loss function that considers variance within literatures and variance between literatures, wherein the process of training the multi-task model converges by minimizing the loss function.

12. The system according to claim 11, wherein:
the extracted study features are expressed by a matrix X with n*d elements, n is a number of the plurality of literatures, and d is a number of the extracted study features;
the extracted weights of risk factors are expressed by a matrix Y with n*m elements, n is a number of the plurality of literatures, and m is a number of the extracted weights of risk factors;
the loss function is set as min_{W}l(X, Y; W)+η+ϵ, wherein
l(X,Y;W)=Σᵢ₌₁ⁿΣⱼ₌₁^{m}½)y_{i,j}−Σₖ₌₁^{d}x_{i,k}*w_{k,j})²,
η is a term indicating the variance between literatures, E is a term indicating the variance within literatures, x_{i,k} represents elements from the matrix X, y_{i,j} represents elements from the matrix Y, and w_{k,j} represents elements from the coefficient matrix W.

13. The system according to claim 12, wherein the term η in the loss function is calculated by using the DerSimonian and Laird method.

14. The system according to claim 8, wherein the study features include study features selected from the group consisting of: impact factor, prediction performance and sample size of a literature.

15. A computer program product comprising a computer readable storage medium having program instructions embodied therewith, wherein the program instructions being executable by a device to cause the device to perform a method comprising:
retrieving a plurality of literatures from one or more databases, wherein each of the plurality of literatures defines a risk prediction model for a same disease;
extracting study features from each of the plurality of literatures;
extracting weights of risk factors in the risk prediction model defined by each of the plurality of literatures from the plurality of literatures; and
calculating adjusted weights of risk factors based on the extracted study features and the extracted weights of risk factors, to form an adjusted risk prediction model.

16. The computer program product according to claim 15, wherein the method further comprising: predicting a risk of the disease for a patient by using the adjusted risk prediction model.

17. The computer program product according to claim 15, wherein calculating the adjusted weights of risk factors comprises:
training a multi-task model using the extracted study features and the extracted weights of risk factors to obtain a coefficient matrix W that follows a Matrix Variate Normal (MVN) distribution;
decomposing the coefficient matrix W to obtain a matrix Ω representing a relationship between risk factors according to the MVN distribution; and
calculating the adjusted weights of risk factors using the matrix Ω.

18. The computer program product according to claim 17, wherein training the multi-task model comprises:
setting a loss function that considers variance within literatures and variance between literatures, wherein the process of training the multi-task model converges by minimizing the loss function.

19. The computer program product according to claim 18, wherein:
the extracted study features are expressed by a matrix X with n*d elements, n is a number of the plurality of literatures, and d is a number of the extracted study features;
the extracted weights of risk factors are expressed by a matrix Y with n*m elements, n is a number of the plurality of literatures, and m is a number of the extracted weights of risk factors;
the loss function is set as min_{W}l(X, Y; W)+η+ϵ, wherein
l(X,Y;W)=Σᵢ₌₁ⁿΣⱼ₌₁^{m}½(y_{i,j}−Σₖ₌₁^{d}*x_{k,j})²,
η is a term indicating the variance between literatures, ϵ is a term indicating the variance within literatures, x_{i,k} represents elements from the matrix X, y_{i,j} represents elements from the matrix Y, and w_{k,j} represents elements from the coefficient matrix W.

20. The computer program product according to claim 19, wherein the term η in the loss function is calculated by using the DerSimonian and Laird method.
